(19) United States
(12) Patent Application Publication
(10) Pub. No.: US 2026/0137627 A1
(43) Pub. Date: May 21, 2026

(54) ENCAPSULATION OF POORLY WATER SOLUBLE COMPOUNDS USING OSA-MODIFIED NON-STARCH POLYSACCHARIDES

(71) Applicant: Kansas State University Research Foundation, Manhattan, KS (US)

(72) Inventor: Yong-Cheng Shi, Manhattan, KS (US)

(21) Appl. No.: 18/705,762

(22) PCT Filed: Oct. 28, 2022
(86) PCT No.: PCT/US2022/078858

§ 371 Date: Apr. 29, 2024

Related U.S. Application Data
(60) Provisional application No. 63/273,548, filed on Oct. 29, 2021.

(51) Int. Cl. A61K 9/48; A61K 9/107

(52) U.S. Cl. CPC A61K 9/4866; A61K 9/107; A61K 9/4833

(57) ABSTRACT

Compounds encapsulated with octenylsuccinic anhydride modified non-starch polysaccharides and methods of forming the encapsulated compounds are provided. Poorly water-soluble compounds, especially pharmaceuticals, are solubilized within an oil phase, which is then formed into an emulsion with an aqueous phase comprising the OSA modified non-starch polysaccharide. The resulting emulsion can be dried, such as through spray drying, for form a powder comprising the encapsulated compound.

### CROSS-REFERENCE TO RELATED APPLICATION

The present application claims the benefit of U.S. Provisional Patent Application No. 63/273,548, filed Oct. 29, 2021, which is incorporated by reference herein in its entirety.

### BACKGROUND OF THE INVENTION

### Field of the Invention

Generally, the present invention is directed toward poorly water-soluble compositions encapsulated with octenylsuccinic anhydride (OSA) modified non-starch polysaccharides and methods of forming the encapsulated compounds. It has been discovered that poorly water-soluble compounds, especially pharmaceutical compounds, can be solubilized within an oil phase, which is then formed into an emulsion with an aqueous phase comprising the OSA modified non-starch polysaccharides. The resulting emulsion can be dried, such as through spray drying, and a high-load powder comprising the encapsulated compound recovered.

### Description of the Prior Art

The Biopharmaceutics Classification System (BCS) is a scientific framework, first introduced in 1995, categorizing drug substances according to their water solubility and intestinal membrane permeability. According to the BCS, drug substances are divided into high/low solubility and permeability classes as follow: Class I (high solubility and high permeability), Class II (low solubility and high permeability), Class III (high solubility and low permeability), and Class IV (low solubility and low permeability).

There is great progress in drug discovery and research, but many new drugs fail to be commercially successful due to unacceptable safety (risk-to-benefit ratio), lack of efficacy, formulation, and market. Major reasons for the failure of new drug candidates are poor water solubility and modest absorption, which relates to many issues such as low bioavailability and increased cost of the drug product. Therefore, it is crucially important to enhance the solubility performance of poorly water-soluble drugs in formulation development.

Fenofibrate (FF), a poorly water soluble (<0.5 mg/L) and highly lipophilic drug, belongs to BCS class II drugs with good permeability but low oral bioavailability. FF in a hard gelatin capsule was originally launched in 1975 with the maximum bioavailability 60%. In the global market, FF (marketed as Tricor®, Lipofen® and Fenoglide®) has been widely used to reduce the levels of low-density lipoprotein-cholesterol and triglyceride, and raise high-density lipoprotein-cholesterol levels in blood, lowering the risk of cardiovascular disease, type 2 diabetes, and renal disease. Pharmaceutical drug performance could be controlled and better designed depending on its molecular structure, and physical state (crystalline or amorphous), where amorphous drugs exhibit a higher dissolution rate than the crystalline substances but worse stability. One type of formulation developed for FF (Lipofen®) is a hard gelatin capsule containing a mixture of a lipid and FF with hydroxypropyl methylcellulose. However, lipid formulations may leach into and interact with the capsule shells, causing brittleness or softness of the capsule shell, leakage of the filling and precipitation of the drug. In oral drug delivery, the drug and the drug carrier have to pass through the stomach with its low pH, which tends to affect the drug stability and the drug solubility as well as the properties of the drug carrier system.

A solid form of self-microemulsifying drug delivery system (solid SEMDDS, a lipid-based drug delivery) has been developed with solid carrier to improve the oral bioavailability of FF. Solid SMEDDS is an anhydrous system consisting of natural or synthetic oil(s), surfactant(s) and cosurfactant(s) or cosolvent(s) incorporated with the lipophilic drug in suitable proportions. However, there exist some limitations associated with SMEDDS. Only small molecule surfactants can be used to prepare SMEDDS. As the formulations of the emulsion always include a great amount of surfactant and co-surfactant, which may cause hemolysis or histopathological alterations of the tissue, disrupt normal membrane structure and may thus lead to cytotoxicity.

Therefore, a need exists in the art for a delivery system for poorly water-soluble compounds, especially pharmaceutical compounds, that preserve the stability and improve the bioavailability of the compound.

### SUMMARY OF THE INVENTION

According to one embodiment of the present invention there is provided an encapsulated composition comprising a poorly water-soluble compound, an oil, and an octenylsuccinic anhydride (OSA) modified non-starch polysaccharide.

According to another embodiment of the present invention there is provided an emulsion comprising a poorly water-soluble compound that is dispersed in an oil phase and an aqueous phase comprising an octenylsuccinic anhydride (OSA) modified non-starch polysaccharide.

According to yet another embodiment of the present invention there is provided a method of forming an encapsulated composition. The method comprises forming an oil phase comprising a poorly water-soluble compound solubilized in an oil. An aqueous phase comprising an octenylsuccinic anhydride (OSA) modified non-starch polysaccharide dissolved in water is also formed. A quantity of the oil phase and a quantity of the aqueous phase are mixed to form an emulsion. The emulsion is then dried to form a powder comprising particles of the poorly water-soluble compound encapsulated in the OSA modified non-starch polysaccharide. The poorly water-soluble compound comprises at least 1% by weight of the powder.

### DETAILED DESCRIPTION OF THE PREFERRED EMBODIMENT

According to one embodiment of the present invention, a micro-emulsion system is formulated for oral administration to improve the solubility and bioavailability of poorly water-soluble drugs, such as fenofibrate (FF), in high-load drug delivery (e.g., 10% in the non-water fraction). Octenylsuccinic anhydride (OSA) modified non-starch polysaccharide, such as phytoglycogen, is used as an emulsifier and solid carrier to form an encapsulated powder.

According to an embodiment of the present invention there is provided an encapsulated composition. The composition comprises a compound that exhibits poor water solubility, an oil, and an octenylsuccinic anhydride (OSA) modified non-starch polysaccharide. In one or more embodiments, the weight ratio of the weight ratio of the OSA modified non-starch polysaccharide to oil is at least 0.1:1, at least 0.2:1, or at least 0.25:1 and/or less than 1:3, less than 1:2, or less than 1:1.5. In one or more embodiments, the weight ratio of the OSA modified non-starch polysaccharide to oil is from about 0.1:1 to about 1:2, from about 0.02:1 to about 1:1.5, or from about 0.5:1 to about 1:1.25.

In certain embodiments, the poorly water-soluble compound comprises at least 1%, 2%, 3%, 5%, 7%, 9%, or 10% by weight of the encapsulated composition. In other embodiments, the composition comprises from about 1% to about 15%, from about 3% to about 15%, from about 5% to about 12%, or from about 7% to about 10% by weight of the poorly water-soluble compound. In one or more embodiments, the poorly water-soluble compound comprises an active pharmaceutical ingredient (API), such as fenofibrate or curcumin. Although nearly any pharmaceutical compound that exhibits poor water solubility may be used. As used herein, the term “poorly water-soluble compound” refers to any compound that has a solubility in water at 25° C. of 10 g/L or less, and preferably less than 1 g/L, less than 100 mg/L, less than 10 mg/L, or less than 1 mg/L. In one or more embodiments, the poorly water-soluble compound may initially be crystalline. However, the encapsulation process may cause the poorly water-soluble compound to acquire an amorphous form, so that the encapsulated poorly water-soluble compound is amorphous.

In certain embodiments, the oil comprises any oil in which the poorly water-soluble compound can be stably dispersed or dissolved, including both natural and synthetic fatty acid esters. Preferably, the oil is approved for human consumption by the U.S. FDA. In particular embodiments, the oil is a vegetable oil such as peanut oil, soybean oil, olive oil, palm kernel oil, coconut oil, or castor oil. The oil may comprise medium-chain triglycerides (MCT) having an aliphatic tail of 6 to 12 carbon atoms. The oil may also comprise a surfactant such as various glycol esters, especially propylene glycol caprylate available under the name Capryol® 90.

In previous work by the current inventor and described in International Patent Application No. PCT/US2021/029823, filed Apr. 29, 2021, incorporated by reference herein in its entirety, a starch-based drug delivery system was developed. Octenylsuccinic anhydride (OSA) modified starch was used as emulsifier to prepare oil-in-water emulsions of poorly water-soluble drugs, e.g., fenofibrate (FF). This emulsion could be spray-dried to produce particles that were highly loaded with the poorly water-soluble drugs. OSA modified starch is prepared by a dry heating method (Shi and Bai, 2016). Medium-chain triglyceride (MCT) or Capryol® 90 is used as oil phase. MCT is a mixture of medium chain triglycerides, mainly from caprylic (C8) and capric (C10) acids (Marten et al., 2006), whereas Capryol® 90 is propylene glycol monocaprylate type II (Shakeel et al., 2013). The solubility of FF in MCT and in Capryol® 90 is 7.93% and 15.4%, respectively (Hu et al., 2011). The obtained emulsions made by micro-fluidizer and spray-dried powders were characterized for particle size distribution, viscosity, and crystallinity. Differential scanning calorimetry (DSC) and X-ray diffraction (XRD) confirmed that the crystalline state of the drug (FF) was converted to the amorphous state in the spray dried powders. The OSA modified starch was able to emulsify and encapsulate 200% (w/w) of the oil phase (Capryol 90) and obtain spray dried powder with high level of FF (8.2%, w/w) and good flow properties. The technology may be used as a promising approach to improve other poorly water-soluble drugs by dissolving them in an oil phase, emulsification by OS starch, and spray drying. In embodiments of the present invention, OSA-modified non-starch polysaccharides are used in place of OSA-modified starch as described in the '823 Application.

The OSA modified non-starch polysaccharide comprises a non-starch polysaccharide component. As the name suggests, a non-starch polysaccharide component means any polysaccharide component that does not comprise, consist of, or consist essentially of starch. Generally, starch is a natural molecule produced by plants that comprises, consists of, or consists essentially of both amylose and amylopectin. Non-starch polysaccharides can include cellulose, pectins, glucans, gums, mucilages, inulin, and chitin. Exemplary glucans include alpha-glucans such as dextran, glycogen (including phytoglycogen), and pullulan, and beta glucans such as cellulose, chrysolaminarin, curdlan, laminarin, lentian, lichenin, pleuran, and zymosan. Exemplary gums include gum arabic and guar gum. Further, as used herein a non-starch polysaccharide component excludes all starches, especially those derived from corn, potato, wheat, rice, tapioca, sago, sorghum, waxy maize, waxy wheat, waxy potato, and high amylose corn.

In one or more embodiments, the OSA modified non-starch polysaccharide can be prepared by dispersing the polysaccharide in an aqueous medium. In certain embodiments, the solution comprises from about 10% to about 40% by weight, from about 15% to about 35% by weight, or from about 20% to about 30% by weight of the polysaccharide. The dispersion can be agitated until the polysaccharide is dissolved. The pH of the solution can be adjusted to between 7 to 8, preferably about 7.5, using a sodium hydroxide (e.g., 3%) solution. OSA is then added to the pH adjusted solution in an amount of from about 1% to about 25% by weight, from about 2% to about 20% by weight, or from about 3% to about 15% by weight, based upon the weight of the polysaccharide, while maintaining the pH of the solution using NaOH. The reaction between the OSA and polysaccharide is allowed to continue for a sufficient time to achieve the desired conversion. In certain embodiments, the reaction progresses for at least 1 to 10 hours, and preferably about 4 hours. The reaction can be terminated by adjusting the pH to 6 using 1 N HCl. The OSA modified non-starch polysaccharide can be recovered by freeze drying, washed by methanol to remove the unreacted OSA, and dried in a vacuum drier. In one or more embodiments, the OSA modified non-starch polysaccharide can have a degree of OSA substitution of from about 0.001 to about 0.050, from about 0.010 to about 0.040, or from about 0.020 to about 0.030.

According to another embodiment of the present invention there is provided an emulsion comprising a poorly water-soluble compound that is dispersed in an oil phase and an aqueous phase comprising an OSA modified non-starch polysaccharide. The aqueous phase can be prepared by adding a quantity of OSA modified non-starch polysaccharide to water to form a polysaccharide solution comprising from about combining from about 5% to about 35% by weight, from about 7.5% to about 30% by weight, or from about 10% to about 25% by weight of the OSA modified non-starch polysaccharide. The oil phase can be prepared by adding a quantity of the poorly water-soluble compound to the oil to form an oil solution comprising from about 1% to about 25% by weight, from about 2.5% to about 20% by weight, or from about 5% to about 15% by weight of the poorly water-soluble compound. In certain embodiments, the poorly water-soluble compound is provided in a crystalline form, which is then solubilized within the oil. Quantities of the polysaccharide and oil solutions are added together in amounts necessary to give the desired non-starch polysaccharide to oil ratio and then mixed to form the emulsion. In one or more embodiments, the emulsion comprises from about 50% to about 90% by weight, from about 60% to about 85% by weight, or from about 70% to about 80% by weight of the aqueous phase. In one or more embodiments, the emulsion comprises from about 5% to about 40% by weight, from about 10% to about 35% by weight, or from about 15% to about 30% by weight of the oil phase. In certain embodiments, the water phase is the continuous phase of the emulsion and the oil phase is the dispersed phase. Also, in certain embodiments, the mixing step can include a homogenization step.

The resulting emulsion exhibits good physical stability in which the oil phase droplets resist aggregation and separation for several hours. In certain embodiments, the emulsions formed comprise oil phase droplets having a mean droplet size of from about 0.05 to about 5 μm, from about 0.075 to about 2.5 μm, or from about 0.1 to about 1 μm. The mean droplet size can remain stable (e.g., within the foregoing ranges) and no separation of the emulsion into visible discrete layers for at least 1 hour, at least 2 hours, or at least 3 hours at 25° C. after emulsion formation. In certain embodiments, the emulsions exhibit a Brookfield viscosity (spindle #21), at 100 rpm shear rate and 25° C. of from about 2 to about 50 cp, from about 3.5 to about 40 cp, or from about 5 to about 35 cp.

In certain embodiments, the emulsion can be dried, preferably by a spray drying process, in which water is removed and a powder comprising the encapsulated poorly water-soluble compound. It has been observed that the poorly water-soluble compound, which originally exhibited a crystalline morphology is now amorphous in its encapsulated form. Thus, the encapsulated compound, especially when the compound is a pharmaceutical compound, exhibits a higher degree of bioavailability than the original crystalline form of the compound. In certain embodiments, the encapsulated particles exhibit a mean particle size (determined as the mean circle equivalent diameter) of from about 1 to about 100 μm, from about 1 to about 50 μm, from about 2 to about 40 μm, from about 4 to about 25 μm, or from about 7 to about 15 μm. Also, the powder remains physically stable for extended periods of time upon storage at 25° C. in that the amount of the poorly water-soluble compound present within the particles remains relatively unchanged for a period of at least 1 month, or at least 3 months, from formation of the powder.

In certain embodiments, the encapsulated powder is capable of being reconstituted into a stable emulsion upon addition of water.

### EXAMPLES

In these Examples, emulsions and encapsulated powders of fenofibrate (FF) are prepared with medium-chain triglyceride (MCT) and Capryol® 90 oils, respectively. After the drug was loaded, the FF completely dissolved in the MCT or Capryol® 90 oil and was encapsulated in OSA modified polysaccharide resulting in an amorphous form of FF.

### Materials

Fenofibrate (purity≥99%) was purchased from Sigma-Aldrich (St. Louis, MO, USA). Medium-chain triglyceride (MCT, Labrafac Lipophile WL 1349) and Capryol® 90 were obtained from Gattefosse Co. (Paramus, NJ, USA). Octenylsuccinic acid anhydride (OSA) was obtained from Gulf Bayport Chemicals L.P. (Pasadena, TX). Phytoglycogen was isolated from sweet corn. All other chemicals were analytical grade.

### Preparation of OSA Modified Phytoglycogen

Phytoglycogen (100 g) was suspended in distilled water (400 ml) with agitation. The pH of the solution was adjusted to 7.5 by using 3% NaOH. OSA (3% or 15% based on the weight of polysaccharide) was added to the solution while pH was maintained at 7.5 by 3% (wt %) NaOH during the reaction. After 4 hours, the reaction was terminated by adjusting pH to 6 with 1 N HCl. The OSA modified phytoglycogen was recovered by freeze drying, washed by methanol to remove the unreacted OSA and dried in a vacuum drier. The degree of substitution (DS) of OSA modified phytoglycogen was determined by high-performance liquid chromatography (HPLC) as previously described (Qiu et al., 2012).

### Preparation of Emulsions and Spray-Dried Powder

OSA modified phytoglycogen (7.5, 10, 12.5, 15, or 17.5 g) in water (70 g or 60 g) was mixed and heated in a water bath at 60° C. for 6 h until the solutions were clear. Fenofibrate (FF, 7 g or 15 g) was dissolved in the oil phase (100 g) at the concentration approaching the saturation concentration of FF in the oil: 7.93% in MCT and 15.4% in Capryol® 90 (Hu et al., 2011). MCT or Capryol® 90 oil phase (12.5, 15.0, 17.5, 20.0, 22.5, or 30 g) was added into OSA modified phytoglycogen while mixing with a portable homogenizer (Bamix, Mettlen, Switzerland) for 3 min. The mixture was pre-homogenized by a bench-top homogenizer (PRO 350, PRO Scientific Inc., Oxford, CT) at 5000 rpm for 10 min, and further homogenized by a micro-fluidizer (M-110P, Microfluidics, Newton, MA) for 3 passes at 30,000 psi. Particle size was measured 1, 2, 4, and 20 h after preparation of the emulsion by a laser diffraction particle size analyzer (LA-910, HORIBA, Ltd., Tokyo, Japan).

Each emulsion was dried in a spray dryer (Mini Spray Dryer B-290, BUCHI Corporation, New Castle, DE USA) operated at an inlet temperature of 160° C. and outlet temperature of 100° C., aspirator 100%, and feed rate 15 ml/min. The collected spray dried powder was packed in a sealed glass bottle and stored at room temperature for further analysis.

Note, phytoglycogen was used in the examples but other polysaccharides such as gum arabic and guar gum can be used.

### Characterization of Emulsion and Encapsulated Powder

Droplet size distribution of emulsions is determined using the laser scattering particle size distribution analyzer (LA-910, Horiba, Japan). All samples are performed in duplicate. The volume mean diameter is used to express the particle size and the width of particle size distribution.

The viscosity of the emulsions before and after homogenized by a micro-fluidizer is measured using a viscometer (DV-II+Pro, Brookfield, Middleboro, MA, USA). A shear rate 100 rpm is chosen, and tests are carried out at room temperature.

The amount of FF in the spray-dried encapsulated powder is determined by HPLC (Agilent 1100 series, Waldbroonn, Germany) equipped with a Phenomenex Kinetex Cis column (Torrance, CA) with 5 μm particle size. A mixture of 70% acetonitrile and 30% water is used as the mobile phase maintained at 25° C. during analysis. The flow rate is 1.0 ml/min, the injection volume is 10 μL and the detection wavelength is set at 286 nm. For FF standard curve, FF (0.1, 0.5, 1.0, 2.0 and 5.0 mg) is weighed, filled with 5 mL of ethanol, and analyzed by HPLC.

Each spray-dried encapsulate (0.1 g) is re-suspended in 0.9 mL of distilled water. An appropriate enzyme is added into slurry and placed into a water bath at 60° C. for 15 min. The emulsion (1 mL) is cooled to 25° C., and 9 mL of methanol is mixed with the emulsion for extraction of FF in the encapsulates. The mixture is centrifuged at 3000 g for 15 min, and the supernatant is filtered by nylon filter membranes (0.45 μm) and analyzed by HPLC. The concentration of FF is determined from the peak area and the amount of FF is calculated from FF standard curve.

Particle size distribution of spray-dried powder is determined using a microscopic image analyzer (Morphologi G3 instrument, Malvern Panalytical Inc., Westborough, MA, USA). Circle equivalent (CE) diameter, high sensitivity (HS) circularity, convexity and elongation of each spray-dried powder are measured and recorded. CE diameter is the diameter of a circle with the same area as the projected area of the particle image. HS circularity has values in the range of 0-1, which a perfect circle has a circularity of 1 while a very “spiky” or narrow elongated object has a circularity value closer to 0. Convexity also has values in the range of 0-1. A smooth shape has a convexity of 1 while a very “spiky” or narrow elongated object has a circularity value closer to 0. Elongation is defined as [1-width/length], which also has values in the range from 0 to 1. A shape such as a circle or square has an elongation value of 0, while a rod has a high elongation (manual).

The density of the encapsulated powder samples is measured using a helium gas pycnometer (AccuPyc 111340, Micromeritics, Norcross, GA, USA), and is calculated from the weight and particle volume. The averages of three measurements are calculated and reported.

The angle of repose is measured by a Hosokawa powder tester (Micron powder systems, Summit, NJ). The sample is poured into the funnel at a stable feeding by vertical vibration. After pouring the samples, the height of the cone is measured, and the angle of repose is calculated using the following relationship:

$$θ = arctan ( 2 H / D ) \qquad ( 1 )$$

Spray-dried encapsulates (0.1 g) are dispersed with 10 mL of distilled water by handshaking, and then the solution was shaken for 10 min. The reconstituted emulsion is stored at room temperature after 3 h. Droplet size distribution of re-suspension emulsions is determined using the laser scattering particle size distribution analyzer as described above.

The physical state of the spray-dried emulsions samples is obtained with an X-ray diffractometer (APD 3520, Philips, Netherlands). The instrument is operated at 35 kV, 20 mA with Cu-Kα radiation, a theta-compensating slit, and a diffracted beam monochromator. Data are recorded between the diffraction angles (2θ) of 2° and 35°.

Differential scanning calorimetry (DSC) measurement of spray dried drug powder is studied with the TA instrument Q200 V24.4 (New Castle, DE, USA) in a range of −20 to 120° C. under nitrogen flow of 70 mL/min. The spray-dried emulsions powder (3-5 mg) is accurately weighed into DSC stainless steel pan. An empty pan is used as a reference. The heating rate of 10° C./min is used.

Stability tests as mentioned above (level of FF in spray-dried powder, particle size and shape, density, flow properties, reconstitution, XRD and DSC) for all spray-dried encapsulates are performed after the storage at room temperature for 3 months.

Analysis of variance is conducted using a Statistical Analysis System (SAS, version 9.3 for Windows, SAS Institute, Cary, NC, USA). Least significant differences for comparison of means are computed at p<0.05.

1. An encapsulated composition comprising a poorly water-soluble compound, an oil, and an octenylsuccinic anhydride (OSA) modified non-starch polysaccharide.

2. The encapsulated composition of claim 1, wherein the OSA modified non-starch polysaccharide comprises an OSA modified glucan.

3. The encapsulated composition of claim 2, wherein the OSA modified glucan comprises OSA modified phytoglycogen.

4. The encapsulated composition of claim 1, wherein the weight ratio of the OSA modified non-starch polysaccharide to oil is at least 0.1:1.

5. (canceled)

6. The encapsulated composition of claim 1, wherein the poorly water-soluble compound is an active pharmaceutical ingredient in an amorphous form.

7. (canceled)

8. The encapsulated composition of claim 1, wherein the oil is a vegetable oil or a nonionic water-insoluble surfactant.

9. The encapsulated composition of claim 8, wherein the vegetable oil comprises peanut oil, soybean oil, olive oil, palm kernel oil, coconut oil, or castor oil, and wherein the nonionic water-insoluble surfactant comprises propylene glycol caprylate.

10. (canceled)

11. (canceled)

12. The encapsulated composition of claim 1, wherein the encapsulated composition is in the form of a powder comprising particles having a mean particle size of from about 1 to about 100 μm, and wherein the amount of the poorly water-soluble compound remains substantially unchanged within the particles making up the powder for a period of at least 1 month at 25° C. following formation of the powder.

13. (canceled)

14. An emulsion comprising a poorly water-soluble compound that is dispersed in an oil phase and an aqueous phase comprising an octenylsuccinic anhydride (OSA) modified non-starch polysaccharide.

15. The emulsion of claim 14, wherein the weight ratio of the OSA modified non-starch polysaccharide to oil is from about 0.1:1 to about 1:2, wherein the aqueous phase comprises from about 5% to about 35% by weight of the OSA modified non-starch polysaccharide, and wherein the oil phase comprises from about 1% to about 25% by weight of the poorly water-soluble compound.

16. (canceled)

17. (canceled)

18. The emulsion of claim 14, wherein the emulsion comprises droplets of the oil phase having a mean droplet size of from about 0.05 to about 5 μm, and wherein the mean droplet size remains stable after emulsion formation for at least 1 hour at 25° C.

19. (canceled)

20. (canceled)

21. The emulsion of claim 14, wherein the emulsion comprises from about 50% to about 90% by weight of the aqueous phase and from about 5% to about 40% by weight of the oil phase.

22. The emulsion of claim 14, wherein the OSA modified non-starch polysaccharide comprises an OSA modified glucan.

23. The emulsion of claim 22, wherein the OSA modified glucan comprises OSA modified phytoglycogen.

24. A method of forming an encapsulated composition comprising:
forming an oil phase comprising a poorly water-soluble compound solubilized in an oil;
forming an aqueous phase comprising an octenylsuccinic anhydride (OSA) modified non-starch polysaccharide dissolved in water;
mixing a quantity of the oil phase and a quantity of the aqueous phase to form an emulsion; and
drying the emulsion to form a powder comprising particles of the poorly water-soluble compound encapsulated in the OSA modified non-starch polysaccharide, wherein the poorly water-soluble compound comprises at least 1% by weight of the powder.

25. (canceled)

26. The method of claim 24, wherein the oil is a vegetable oil or nonionic water-insoluble surfactant, and wherein the poorly water-soluble compound is a pharmaceutical compound, and wherein the OSA modified non-starch polysaccharide is an OSA modified glucan.

27. The method of claim 24, wherein the oil phase comprises from about 1% to about 25% by weight of the poorly water-soluble compound, and wherein the aqueous phase from about 5% to about 35% by weight of the OSA modified non-starch polysaccharide.

28. The method of claim 24, wherein the emulsion comprises droplets of the oil phase having a mean droplet size of from about 0.05 to about 5 μm, and wherein the powder comprises particles having a mean particle size of from about 1 to about 100 μm.

29. The method of claim 24, wherein the emulsion comprises from about 50% to about 90% by weight of the aqueous phase and from about 5% to about 40% by weight of the oil phase.

30. The method of claim 24, wherein the poorly water-soluble compound is in a crystalline form prior to being solubilized in the oil, and wherein the poorly water-soluble compound is in an amorphous form when encapsulated in the OSA modified non-starch polysaccharide.

31. (canceled)
